(19) United States
(12) Patent Application Publication
(10) Pub. No.: US 2020/0253493 A1
(43) Pub. Date: Aug. 13, 2020

(54) MINIATURE IMPLANTABLE WIRELESS PRESSURE SENSOR

(71) Applicant: THE REGENTS OF THE UNIVERSITY OF CALIFORNIA, Oakland, CA (US)

(72) Inventors: Mark Bachman, Irvine, CA (US); Sarkis Babikian, Irvine, CA (US); Guann-Pyng Li, Irvine, CA (US)

(21) Appl. No.: 16/639,366

(22) PCT Filed: Aug. 16, 2018
(86) PCT No.: PCT/US18/46806

§ 371 Date: Feb. 14, 2020

Related U.S. Application Data
(60) Provisional application No. 62/546,455, filed on Aug. 16, 2017.

(51) Int. Cl. A61B 5/03; A61B 3/16; A61B 5/00; A61B 5/05

(52) U.S. Cl. CPC A61B 5/031; A61B 3/16; A61B 5/0031; A61B 2562/14; A61B 2562/0247; A61B 2562/0223; A61B 5/05

(57) ABSTRACT

A miniature wireless pressure sensor has an inductor and a capacitor. The inductor and the capacitor form a L-C resonator with a resonate frequency. The inductor's inductance is affected by a slidable electro-magnetic element. When an outside pressure is applied onto the element, it causes the element to move and such movement changes the inductance of the inductor. Because of that, the resonate frequency is changed. Therefore, the change in resonate frequency indicates a change in the outside pressure. The L-C resonator is calibrated to correlate with the outside pressure. Such a miniature wireless pressure sensor facilitates the monitoring of physiological pressure in different part of human body such as eyes and cranium.

### CROSS REFERENCE

This application claims priority to U.S. Patent Application No. 62/546,455, filed on Aug. 16, 2017, the specification(s) of which is/are incorporated herein in their entirety by reference.

### FIELD OF INVENTION

The present invention is related to a wireless pressure sensor. More particularly, it is related to a miniature implantable wireless pressure sensor. The pressure sensor facilitates the monitoring of physiological pressures in different parts of human body to provide real-time monitoring for applications such as glaucoma, intracranial hypertension and other pressure related indications.

### BACKGROUND OF THE INVENTION

Recent studies in the management of diseases/injuries such as glaucoma and head trauma have revealed the importance of continuous monitoring of physiological pressure such as intraocular pressure (IOP) and intracranial pressure (ICP). Monitoring of these pressures may make treatment more effective and may facilitate prompt intervention when sudden pressure spikes or unforeseen oscillations occur. Thus, implantable, wireless sensors are crucial to facilitate such continuous monitoring of pressure. These sensors should be small to be less traumatic and easy to implant.

Several methods and devices have been reported for continuous measuring of physiological pressure, and especially for IOP. The reported devices can be categorized into active and passive sensors. Active sensors incorporate batteries and integrated circuit that actively transmit pressure information to a wireless reader, and passive sensors are interrogated by a non-contact external reader.

The passive sensors are advantageous due to having rather simple structures and compatible with miniaturization to scales that are suitable for implantation. Moreover, passive devices are generally preferred since battery technology brings additional size and risk of contamination for the implantable device.

Examples of passive devices include passive radio wave resonators that incorporate a capacitive pressure transducer and can be read by near field magnetic coupling. Capacitive-based pressure sensing is quite common. Most passive devices utilize a resonating circuit that changes its resonate frequency when the capacitance changes.

Capacitive membrane devices rely on a large membrane and a small gap between the membrane and an electrically conducting condenser plate. And thus, they tend to be thin in one dimension, but large in cross section area, like a pancake structure.

This kind of structure makes capacitive-based pressure sensors unsuitable for applications such as glaucoma, intracranial hypertension pressure monitoring.

### SUMMARY OF THE INVENTION

The present invention is about a miniature tube shape wireless pressure sensor (100). The sensor (100) comprises a sensor housing which is a miniature tube (110) having a hollow interior (120), a slidable first end (130) and a fixed second end (140); wherein the first end (130) is a pressure sensing interface.

The pressure sensor also comprises an inductor coil (150) patterned on an exterior side of the tube (110) towards the first end (130) of the tube (110), a capacitor module (160) mounted on the exterior side of the tube (110) towards the second end (140) of the tube (110), wherein the capacitor module (160) and the inductor forms an inductance-capacitance L-C resonator with a resonant frequency.

Besides that, the pressure sensor has an electro-magnetic fluid (170) disposed in the hollow interior (120) of the tube (110) towards the first end (130) of the tube, and an inert gas (180) disposed in the hollow interior (120) of the tube (110) towards the second end (140) of the tube (110).

When an outside pressure is applied to the electro-magnetic fluid (170) through the pressure sensing interface (130), the electro-magnetic fluid (170) slides inside the inductor coil (150) and this movement alters an inductance of the inductor coil (150). When the inductance of the inductor coil (150) is altered by the outside pressure, it causes a change in the resonant frequency of the L-C resonator. The change in the resonant frequency indicates a change in the outside pressure.

### BRIEF DESCRIPTION OF THE DRAWINGS

The features and advantages of the present invention will become apparent from a consideration of following detailed description presented in connection with the accompanying drawings in which:

FIG. 1A-1B are schematic drawings of a pressure sensor (10). FIG. 1A shows a pressure (10) utilizing a movement of a magnetic material (19) under an influence of an external pressure (12). FIG. 1B shows a pressure sensor (10) utilizing the movement of an inductor (13) under the influence of the external pressure (12).

FIG. 2A-2B are schematic drawings showing possible pressure sensor device housing configurations.

FIG. 3A-3B are schematic drawing showing possible Inductor and magnetic material configurations.

FIG. 4 is a perspective view of an embodiment of the wireless pressure sensor (100).

FIG. 5 is a cross section view of a first embodiment of the wireless pressure sensor (100) shown in FIG. 4.

FIG. 6 is a cross section view of a second embodiment of the wireless pressure sensor (100) shown in FIG. 4.

FIG. 7 is a cross section view of a third embodiment of the wireless pressure sensor (100) shown in FIG. 4.

FIG. 8 illustrates a near field interrogation scheme with magnetic coupling.

FIG. 9 illustrates a backscatter radio wave interrogation scheme.

### DETAIL DESCRIPTION OF THE INVENTION

Each of the additional features and teachings disclosed bellow can be utilized separately or in conjunction with other features and teachings to provide a wireless sensor. Representative examples of the embodiments described herein, which examples utilize many of these additional features and teachings both separately and in combination, will now be described in further detail with reference to the attached drawings. This detailed description is merely intended to teach a person of skill in the art further details for practicing preferred aspects of the present teachings and is not intended to limit the scope of the invention. Therefore, combinations of features and steps disclosed in the following detail description may not be necessary to practice the invention in the broad sense and are instead taught merely to particularly describe representative examples of the present teaching.

Moreover, the various features of the representative examples and the dependent claims may be combined in ways that are not specifically and explicitly enumerated to provide additional useful embodiments of the present teachings. In addition, it is expressly noted that all features disclosed in the description and/or the claims are intended to be disclosed separately and independently from each other for original disclosure, as well as for restricting the claimed subject matter independent of the compositions of the features in the embodiments and/or the claims. It is also expressly noted that all value ranges or indications of groups of entities disclose every possible intermediate value or intermediate entity for original disclosure, as well as for restricting the claimed subject matter.

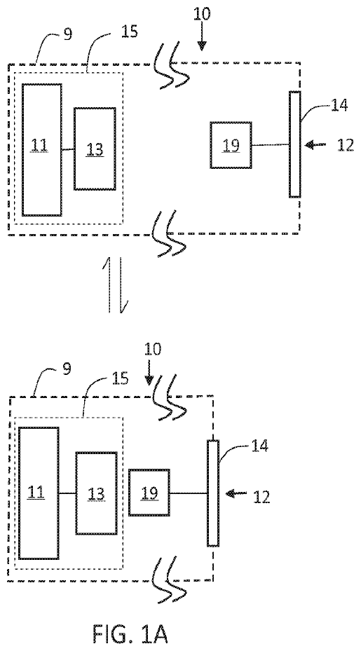

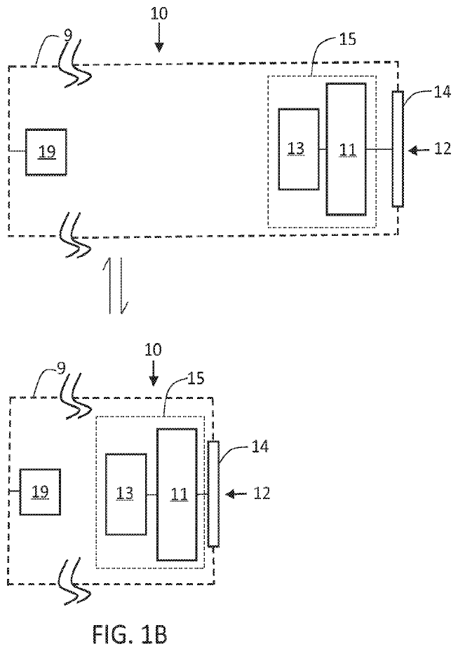

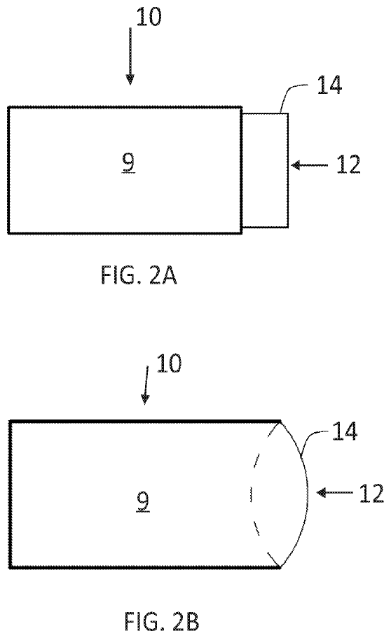

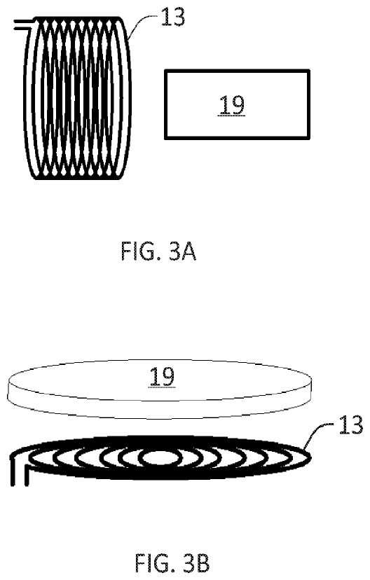

In a broad embodiment, a wireless pressure sensor device (10) comprises a sensor housing (9), a capacitor (11), an inductor (13) and a magnetic material (19). See FIGS. 1A and 1B,

The capacitor (11) and the inductor (13) are operatively connected to form an inductance-capacitance L-C resonator (15) with a first resonance frequency (17). The magnetic material (19) is at an initial distance from the inductor (13). The sensor housing (9) has a displaceable surface (14) biased to be in an initial resting position (8) by a restorative force (6). In the present invention, the size of the sensor device (10) is about 0.1 mm to 5 mm long and 0.1 mm to 5 mm in diameter.

When an external pressure (12) is applied to the displaceable surface (14), the displaceable surface (14) is actuated, thereby creating a shift in the distance between the magnetic material (19) and the inductor (13). The degree of the shift is proportional to the external pressure (12). The shift in distance may be between about 0.01 mm to 4.9 mm. For example, the shift in distance is about 1 mm.

In some embodiments, if the inductor (13) is stationary, then the magnetic material (19) is moveable such that when the displaceable surface (14) is actuated, the magnetic material (19) moves relative to the inductor (13) to create the shift in distance. As an example, the inductor (13) could remain stationary by being affixed to an interior of the housing (9).

In some embodiments, if the magnetic material (19) is stationary, then the inductor (13) is moveable such that when the displaceable surface (14) is actuated, the inductor (13) moves relative to the magnetic material (19) to create the shift in distance. As an example, the magnetic material (19) could remain stationary by being affixed to an interior of the housing (9).

In some embodiments, the distance shift between the magnetic material (19) and the inductor (13), which is proportional to the external pressure (12), causes a change in the inductance of the inductor (13). Which in turn changes the resonance frequency (17) of the L-C resonator (15). Thereby allowing for detection and measurement of said external pressure (12). The pressure difference detected with this technology could be between 0.5 mmHg to 10 mmHg or about.

The various embodiments provided herein are generally directed to implantable wireless pressure sensors that facilitate the monitoring of physiological pressures in different parts of the human body to provide monitoring for applications such as glaucoma, intracranial hypertension and other pressure related indications. The embodiments are in the form of a small wireless pressure sensor that is implanted in the human body to continuously and directly measure physiological pressures, such as intraocular pressure (IOP), intracranial pressure (ICP) or other pressure. The pressure sensor includes an electric coil and a slidable element that moves when pressure is changed. This movement effectively changes the inductance of the coil. The pressure sensor which has electrical properties of inductance and capacitance acts as L-C resonator. The L-C resonator can be interrogated using external electromagnetic radiation, and thus requires no internal energy source for operation.

The present disclosure provides a wireless pressure sensor utilizing a variable inductive transducer. It comprises an inductor-capacitor circuit also know as L-C resonator tank. The inductor component of the resonator is configured such that its value changes with the ambient pressure. Thus, a change in the ambient pressure cause a change in the inductance value of the circuit, which in turn causes a change in the resonance frequency of the L-C resonator.

Features of the embodiment includes a coil forming an inductor and a movable element that moves in the response to a change in ambient pressure. The movable element is composed of a material that changes the effective inductance of the coil.

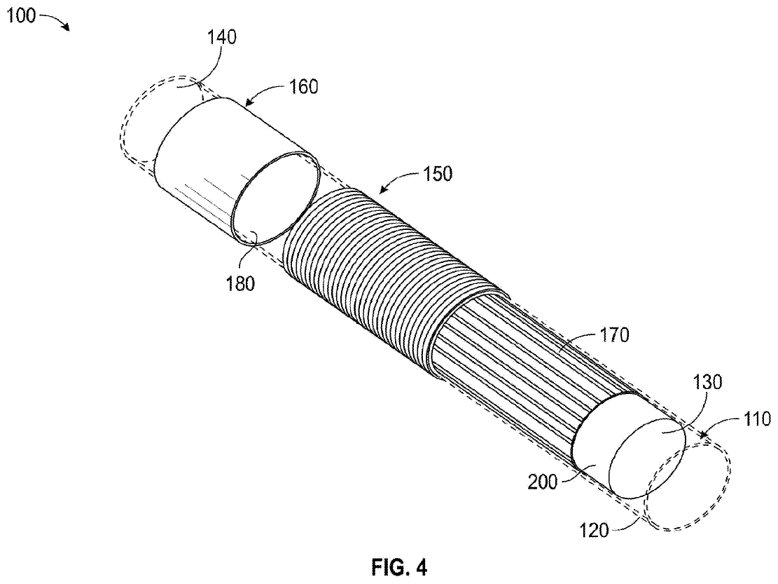

As shown in FIG. 4, the exemplary sensor utilizing a variable inductive transducer includes an inductor coil patterned on the wall of a capillary tube. The capillary tube is closed on one end and open on the other end, which serves as the pressure sensing interface or port. The inductor coil is patterned on the opening end of the tube. The capillary tube is filled with an electro-magnetic fluid on the opening end. A capacitor module is mounted towards to the closed end. The inductor and the capacitor form an L-C resonator.

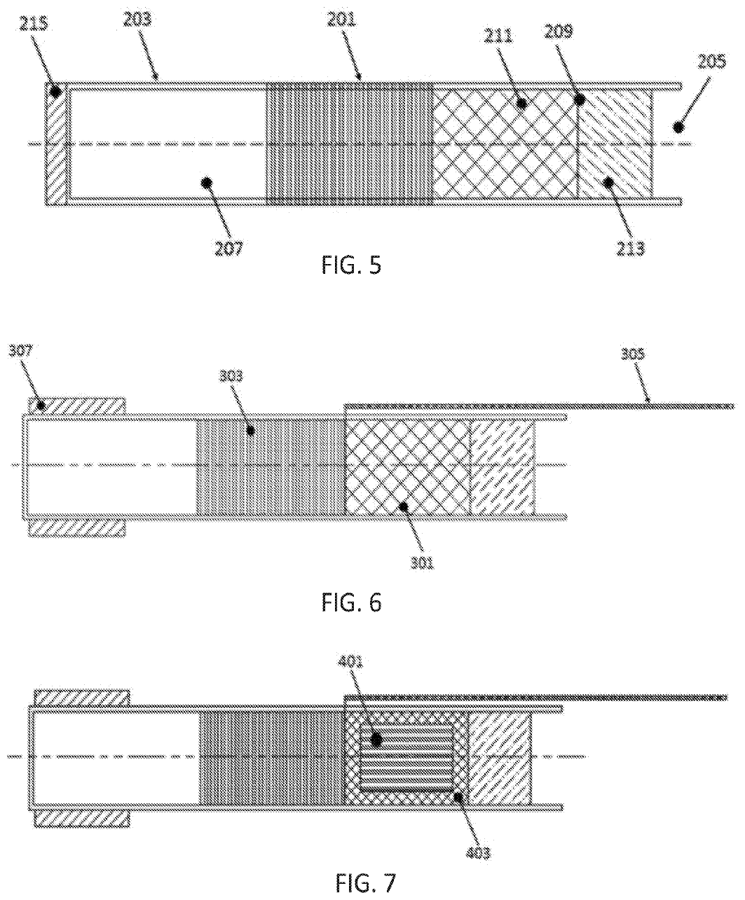

Typical embodiments of the inductive pressure transducer are illustrated in FIGS. 5, 6 and 7. The first embodiment of the sensor is shown in FIG. 5. The sensor consists of an inductor coil (201) patterned or fabricated on the walls of a micro capillary tube or a microchannel (203). The capillary tube (203) is made of a dielectric, chemically inert and flexible material such as fused silica or polymer tubing. The coil (201) may be pattern on either the out walls or the inner walls of the capillary tube (203). The capillary tube (203) is closed on one end and open on the other end (205). The open end serves as a pressure sensing interface or port. The induction coil (201) is patterned towards the open end (205).

The capillary tube (203) is filled with a gas (207) which serves as a pneumatic compression spring. The interface between the inert gas and external environment is comprised of one or two incompressible fluid volumes or regions. A fluid region (209) may have special electrical or magnetic properties that may alter the inductance value of the coil (201) when pushed into the region of the capillary tube (203) overlapped with the coil (201). Fluid (211) can be impregnated with soft magnetic nano-particles such as ferrites (iron oxides) or other variants of alloy particles or nano-engineered particles with magnetic properties. In this example, the fluid 211 is called a ferrofluid or magnetic fluid.

A capacitor module (215) is disposed at the closed end of the capillary tube (203) and forms an L-C resonator with the coil (201). In this embodiment, the coil (201) also functions as an antenna of the sensor.

When the magnetic fluid (211) moves inside the coil (201) under the influence of outside pressure, the relative magnetic permeability of the core of the coil (201) is altered by the fluid (211), which in turn alters the inductance of the coil (201) and the resonance frequency of the L-C resonator. A viscous fluid (213) is used to isolate the capillary fluid (211) from the external environment fluid.

In another embodiment, shown in FIG. 6. The fluid (301) may have high electrical conductivity, such as electrolytic fluids, metal salts. The coil (303) is fabricated on the internal walls of the capillary tube. The conductive fluid (301) shunts the turns of the coil (303) as it gets displaced inside the capillary tube and flows over the coil's bare conductor. Thus, the coil (303) is effectively shortened in length. Therefore, the inductance and the L-C resonance frequency is changed.

Also, alternatively, the capacitor (307) of the resonator may be fabricated on the walls of the capillary by deposition of multiple layers of conductive and dielectric materials.

An optional or additional antenna (305) may be added to the device, the geometry of this antenna is defined by the interrogation method discussed below.

In another embodiment as shown in FIG. 7, the fluid core may be replaced with a solid material (401) that may have magnetic or electric properties as per discussion above, and that can move in response to a pressure change. In this case, a viscous bearing (403) may be added.

Similar embodiments may be envisioned utilizing a compressible material instead of a gas filled pneumatic cavity to provide the restoring force against the external pressure.

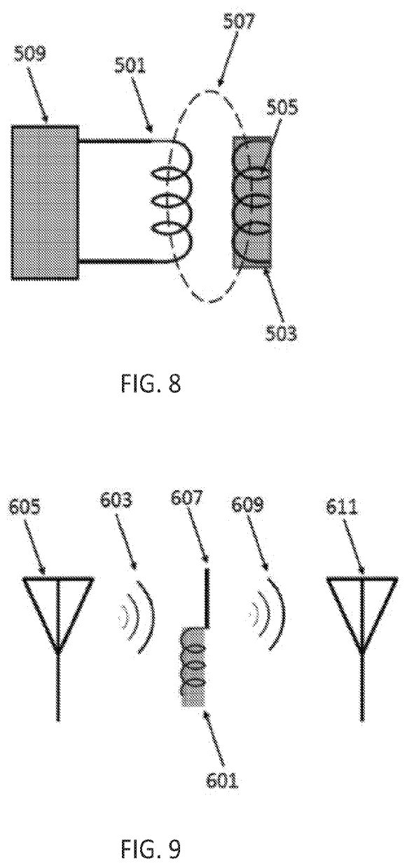

For example, the wireless interrogation of this sensor may be done through two methods utilizing radio frequency signals. FIG. 8 describes a first method of using inductive coupling. In this method, the probe antenna (501) is a loop antenna placed at proximity to the sensor (503). The inductor coil (505) of the sensor (503) may serve as the sensor antenna, or an additional loop antenna may be added to the sensor (503). The near field magnetic line (507) of the probe antenna (501) couples with the sensor antenna (505). Thus, an inductive coupling between the probe (501) and sensor (503) is established. A change in pressure is seen as a change in the electrical response at the probe terminals. This may be directly read by the electronic circuitry (509).

In another embodiment, the wireless interrogation can be done using radio frequency signals and the backscattering property of antenna as shown in FIG. 9. In this method, the sensor (601) is interrogated with electromagnetic waves. The incident radio wave (603) (coming from the excitation antenna (605)) on a sensor's antenna (607) causes a current to flow through the sensor's antenna (607) and the LC resonator connected to it. If the radio wave frequency matches with the resonance frequency, a large current would flow in the circuit, which in turn would generate its own electromagnetic radiation. The backscatter wave (609) may be detected by a receiving antenna (611) on the probe side. Thus, the resonance frequency, which is related to the pressure value, of the sensor (601) is determined. The excitation and receiving antenna may be physically one antenna or two separate antennas.

These examples are illustrative of various embodiments and additional features that are afforded by the wireless pressure sensor and are not intended to represent an exhaustive list of features. The example embodiments provided herein, however, are merely intended as illustrative examples and not limiting in any way.

All features, elements, components, functions, and steps described with respect to any embodiment provided herein are intended to be freely combinable and substitutable with those from other embodiment. If a certain feature, element, component, function or step is described with respect to only one embodiment, then it should be understood that that feature, element, component, function or step can be used with every other embodiment described herein unless explicitly stated otherwise. This paragraph therefore serves as antecedent basis and written support for the introduction of claims, at any time, that combine features, elements, components, functions and steps from different embodiments, or that substitute features, elements, functions, and steps from one embodiment with those of another, even if the following description does not explicitly state, in a particular instance, that such combination or substitutions are possible. Express recitation of every possible combination and substitution is overly burdensome, especially given that the permissibility of each of each such combination and substitution will be readily recognized by those of ordinary skill in the art upon reading this description.

In many instance entities are described herein as being coupled to other entities. The terms “coupled” and “connected” or any of their forms are used interchangeable herein and, in both cases, are generic to the direct coupling of two entities.

While the embodiments are susceptible to various modifications and alternative forms, specific examples thereof have been shown in the drawings are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments maybe recited in or added to the claims, as well as negative limitations that define the inventive scope of the claims by features, functions, steps or elements that are not within that scope.

1. A wireless pressure sensor device (10) comprising:
(a) a sensor housing (9) having a displaceable surface (14) biased to be in a resting position (8) by a restorative force (6);
(b) a capacitor (11) disposed in or on the sensor housing (9);
(c) an inductor (13) disposed in or on the sensor housing (9), wherein the inductor (13) and the capacitor (11) are operatively connected to form an inductance-capacitance L-C resonator (15) with a first resonance frequency (17); and
(d) a magnetic material (19) disposed in the sensor housing (9), wherein the magnetic material (19) is at a distance from the inductor (13);
wherein when an external pressure (12) is applied to the displaceable surface (14), the displaceable surface (14) is actuated, thereby creating a shift in the distance between the magnetic material (19) and the inductor (13);
wherein the distance shift between the magnetic material (19) and the inductor (13) causes a change in the inductance of the inductor (13), which in turn changes the resonance frequency (17) of the L-C resonator (15), thereby allowing for detection and measurement of said external pressure (12).

2. The pressure sensor (10) of claim 1, wherein the inductor (13) is stationary and the magnetic material (19) is moveable such that when the displaceable surface (14) is actuated, the magnetic material (19) moves relative to the inductor (13) to create the shift in distance.

3. The pressure sensor (10) of claim 1, wherein the magnetic material (19) is stationary and the inductor (13) is moveable such that when the displaceable surface (14) is actuated, the inductor (13) moves relative to the magnetic material (19) to create the shift in distance.

4. The pressure sensor (10) of claim 1, wherein a state of the magnetic material (19) is a liquid.

5. The pressure sensor (10) of claim 1, wherein a state of the magnetic material is a solid.

6. The pressure sensor (10) of claim 1, wherein a shape of the magnetic material is a disk or a tube.

7. The pressure sensor (10) of claim 1, wherein a shape of the inductor (13) is a helical coil.

8. The pressure sensor of claim 1, wherein a shape of the inductor (13) is a spiral disk.

9. The pressure sensor (10) of claim 1, wherein a shape of the sensor housing is a tube.

10. The pressure sensor (10) of claim 1, wherein a size of the sensor is at millimeter scale.

11. The pressure sensor of claim 1, wherein the resonance frequency (17) is measurable wirelessly by magnetic coupling or by backscattered radio wave.

12. The sensor (10) of claim 1, wherein the restorative force (6) component is a spring or an inert gas.

13. The sensor (10) of claim 1, wherein the capacitor is disposed on an exterior surface of the sensor housing.

14. The sensor (10) of claim 1 further comprises an antenna.

15. The sensor (10) of claim 1 detects and measures a fluid pressure.

16. A miniature tube shape wireless pressure sensor (100), the sensor (100) comprises:
(a) a sensor housing which is a miniature tube (110) having a hollow interior (120), a slidable first end (130) and a fixed second end (140); wherein the first end (130) is a pressure sensing interface;
(b) an inductor coil (150) patterned on an exterior side of the tube (110) towards the first end (130) of the tube (110);
(c) a capacitor module (160) mounted on the exterior side of the tube (110) towards the second end (140) of the tube (110), wherein the capacitor module (160) and the inductor forms an inductance-capacitance L-C resonator with a resonant frequency;
(d) an electro-magnetic fluid (170) disposed in the hollow interior (120) of the tube (110) towards the first end (130) of the tube; and
(e) an inert gas (180) disposed in the hollow interior (120) of the tube (110) towards the second end (140) of the tube (110);
wherein when an outside pressure is applied to the electro-magnetic fluid (170) through the pressure sensing interface (130), the electro-magnetic fluid (170) slides inside the inductor coil (150) and this movement alters an inductance of the inductor coil (150); and
wherein when the inductance of the inductor coil (150) is altered by the outside pressure, it causes a change in the resonant frequency of the L-C resonator, wherein, the change in the resonant frequency indicates a change in the outside pressure.

17. The pressure sensor (100) of claim 16, wherein the L-C resonator is calibrated to correlate with the outside pressure.

18. The pressure sensor (100) of claim 16, wherein the inductor coil (150) is also serve as an antenna of the wireless sensor.

19. The pressure sensor (100) of claim 16, wherein the tube comprises of dielectric, chemically inert and flexible material.

20. The pressure sensor (100) of claim 19, wherein the tube is constructed at least partly from fused silica or polymer tubing.

21.-24. (canceled)
